(19) United States
(12) Patent Application Publication
(10) Pub. No.: US 2015/0272659 A1
(43) Pub. Date: Oct. 1, 2015

(54) TWO STAGE TRIGGER, CLAMP AND CUT BIPOLAR VESSEL SEALER

(71) Applicant: ETHICON ENDO-SURGERY, INC., CINCINNATI, OH (US)

(72) Inventors: Chad P. Boudreaux, Cincinnati, OH (US); John M. Sarley, Mason, OH (US); Gregory A. Trees, Loveland, OH (US); Catherine A. Corbett, Cincinnati, OH (US)

(73) Assignee: ETHICON ENDO-SURGERY, INC., CINCINNATI, OH (US)

(21) Appl. No.: 14/227,699

(22) Filed: Mar. 27, 2014

(51) Int. Cl. A61B 18/14

(52) U.S. Cl. CPC A61B 18/1445; A61B 2018/0063

(57) ABSTRACT

In various embodiments, a surgical instrument is disclosed. In one embodiment, the surgical instrument comprises a handle assembly. The handle assembly comprises a closure trigger, a push plate, a clamp plate, and a firing plate. Actuation of the closure trigger rotates the push plate. Rotation of the push plate to a first rotation rotates the clamp plate and rotation between the first rotation and a second rotation rotates the firing plate. A shaft assembly is coupled to the handle assembly. An end effector is coupled to the shaft assembly. The end effector comprises a jaw assembly. The jaw assembly defines a longitudinal slot. Rotation of the clamp plate pivotally moves a first jaw member from an open position to a closed position relative to a second jaw member. A cutting member is deployable within the longitudinal slot.

### BACKGROUND

The present disclosure is related generally to electrosurgical devices with various mechanisms for clamping and treating tissue. In particular, the present disclosure is related to electrosurgical devices with two stage triggers.

While several devices have been made and used, it is believed that no one prior to the inventors has made or used the device described in the appended claims.

### SUMMARY

In one embodiment, a surgical instrument is disclosed. The surgical instrument comprises a handle assembly, a shaft assembly, and an end effector. The shaft assembly comprises a closure trigger, a push plate coupled to the closure trigger, a clamp plate coupled to the push plate, and a firing plate coupled to the push plate. Actuation of the closure trigger rotates the push plate. Actuation of the closure trigger to a first rotation rotates the clamp plate. Actuation of the closure trigger between the first rotation and a second rotation rotates the firing plate. The shaft assembly comprises a proximal end and a distal end. The shaft assembly is coupled to the handle assembly at the proximal end. The end effector is coupled to the distal end of the shaft assembly. The end effector comprises a jaw assembly having a proximal end and a distal end. The jaw assembly comprises a first jaw member and a second jaw member. The first and second jaw members define a longitudinal slot. Rotation of the clamp plate pivotally moves the first jaw member from an open position to a closed position relative to the second jaw member. A cutting member is deployable within the longitudinal slot. Rotation of the firing plate advances the cutting member distally within the longitudinal slot.

In one embodiment, a surgical instrument is disclosed. The surgical instrument comprises a handle assembly, a shaft assembly, and an end effector. The handle assembly comprises a closure trigger, a cam shaft plate coupled to the closure trigger, a plunger comprising a plunger pin configured to interface with the cam path. The cam shaft plate defines a cam path having a detent. Proximal actuation of the closure trigger rotates the cam shaft plate into contact with the plunger pin. The plunger pin follows the cam path to interface with the detent. The detent maintains the cam shaft plate and the closure trigger at a first rotation. The shaft assembly comprises a proximal end and a distal end. The shaft assembly is coupled to the handle assembly at the proximal end. The end effector is coupled to the distal end of the shaft assembly. The end effector comprises a jaw assembly having a proximal end and a distal end. The jaw assembly comprises a first jaw member and a second jaw member. The first and second jaw members define a longitudinal slot. Rotation of the clamp plate pivotally moves the first jaw member from an open position to a closed position relative to the second jaw member. A cutting member is deployable within the longitudinal slot. Rotation of the firing plate advances the cutting member distally within the longitudinal slot.

In various embodiments a surgical instrument is disclosed. The surgical instrument comprises a handle assembly, a shaft assembly, and an end effector. The handle assembly comprises a closure trigger, a push plate coupled to the closure trigger, a clamp plate coupled to the push plate, a firing plate coupled to the push plate, a cam shaft plate coupled to the closure trigger, and a plunger configured to interface with the cam path. Actuation of the closure trigger rotates the push plate. Actuation of the closure trigger to a first rotation rotates the clamp plate. Actuation of the closure trigger between the first rotation and a second rotation rotates the firing plate. The cam shaft plate defines a cam path having a detent. Proximal actuation of the closure trigger rotates the cam shaft plate into contact with the plunger. The plunger follows the cam path to interface with the detent. The detent maintains the cam shaft plate and the closure trigger at a first rotation. The shaft assembly comprises a proximal end and a distal end. The shaft assembly is coupled to the handle assembly at the proximal end. The end effector is coupled to the distal end of the shaft assembly. The end effector comprises a jaw assembly having a proximal end and a distal end. The jaw assembly comprises a first jaw member and a second jaw member. The first and second jaw members define a longitudinal slot. Rotation of the clamp plate pivotally moves the first jaw member from an open position to a closed position relative to the second jaw member. A cutting member is deployable within the longitudinal slot. Rotation of the firing plate advances the cutting member distally within the longitudinal slot.

### FIGURES

The novel features of the embodiments described herein are set forth with particularity in the appended claims. The embodiments, however, both as to organization and methods of operation may be better understood by reference to the following description, taken in conjunction with the accompanying drawings as follows:

FIG. 1 illustrates one embodiment of a surgical instrument comprising a two stage trigger;

FIG. 2 illustrates one embodiment of the surgical instrument of FIG. 1 with a left handle housing removed;

FIG. 3 illustrates one embodiment of the surgical instrument in FIG. 1 in an initial position;

FIG. 4 illustrates one embodiment of a trigger assembly of the surgical instrument in FIG. 1;

FIG. 5 illustrates an exploded view of the surgical instrument of FIG. 1;

FIG. 6 illustrates one embodiment of the surgical instrument of FIG. 1 having a closure trigger in a partially rotated position;

FIG. 7 illustrates one embodiment of a cam path plate and a plunger when the closure trigger is in the partially rotated position of FIG. 6;

FIG. 8 illustrates one embodiment of the surgical instrument of FIG. 1 having the closure trigger rotated to a full clamp;

FIG. 9 illustrates one embodiment of the camp path plate and the plunger when the closure trigger is rotated to the full clamp position;

FIG. 10 illustrates one embodiment of the surgical instrument of FIG. 1 having the plunger in a locked position;

FIG. 11 illustrates one embodiment of the trigger assembly of the surgical instrument of FIG. 1 when the closure trigger is rotated to the full clamp position;

FIG. 12 illustrates one embodiment of the surgical instrument of FIG. 1 having the closure trigger rotated to a fired position;

FIG. 13 illustrates one embodiment of the trigger assembly of the surgical instrument of FIG. 1 in a partial return position;

FIG. 14 illustrates one embodiment of the surgical instrument of FIG. 1 comprising a two-stage bypass switch;

FIG. 15 illustrates one embodiment of the trigger assembly of the surgical instrument of FIG. 1 in a bypass position;

FIG. 16 is a graph illustrating one embodiment of a force to fire the surgical instrument of FIG. 1 from an open to a fully fired position;

FIG. 17 is a graph illustrating one embodiment of a mechanical advantage of the closure trigger as the closure trigger is rotated from an initial position to a full clamp position;

FIG. 18 is a graph illustrating one embodiment of a mechanical advantage of the closure trigger as the closure trigger is rotated from the full clamp position to a fired position;

FIG. 19 is a graph illustrating one embodiment of a clamp load at a jaw assembly of an end effector when the jaw assembly is partially open;

FIG. 20 is a graph illustrating one embodiment of force to fire for a clamping stroke of the surgical instrument of FIG. 1;

FIG. 21 illustrates one embodiment of an energy button;

FIG. 22 illustrates one embodiment of an electrosurgical control circuit;

FIG. 23 illustrates one embodiment of the surgical instrument of comprising an electrosurgical energy system.

### DESCRIPTION

In the following detailed description, reference is made to the accompanying drawings, which form a part hereof. In the drawings, similar symbols and reference characters typically identify similar components throughout the several views, unless context dictates otherwise. The illustrative embodiments described in the detailed description, drawings, and claims are not meant to be limiting. Other embodiments may be utilized, and other changes may be made, without departing from the scope of the subject matter presented here.

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Before explaining the various embodiments of the surgical devices having two stage triggers in detail, it should be noted that the various embodiments disclosed herein are not limited in their application or use to the details of construction and arrangement of parts illustrated in the accompanying drawings and description. Rather, the disclosed embodiments may be positioned or incorporated in other embodiments, variations and modifications thereof, and may be practiced or carried out in various ways. Accordingly, embodiments of the surgical devices with two stage triggers disclosed herein are illustrative in nature and are not meant to limit the scope or application thereof. Furthermore, unless otherwise indicated, the terms and expressions employed herein have been chosen for the purpose of describing the embodiments for the convenience of the reader and are not to limit the scope thereof. In addition, it should be understood that any one or more of the disclosed embodiments, expressions of embodiments, and/or examples thereof, can be combined with any one or more of the other disclosed embodiments, expressions of embodiments, and/or examples thereof, without limitation.

Also, in the following description, it is to be understood that terms such as front, back, inside, outside, top, bottom and the like are words of convenience and are not to be construed as limiting terms. Terminology used herein is not meant to be limiting insofar as devices described herein, or portions thereof, may be attached or utilized in other orientations. The various embodiments will be described in more detail with reference to the drawings.

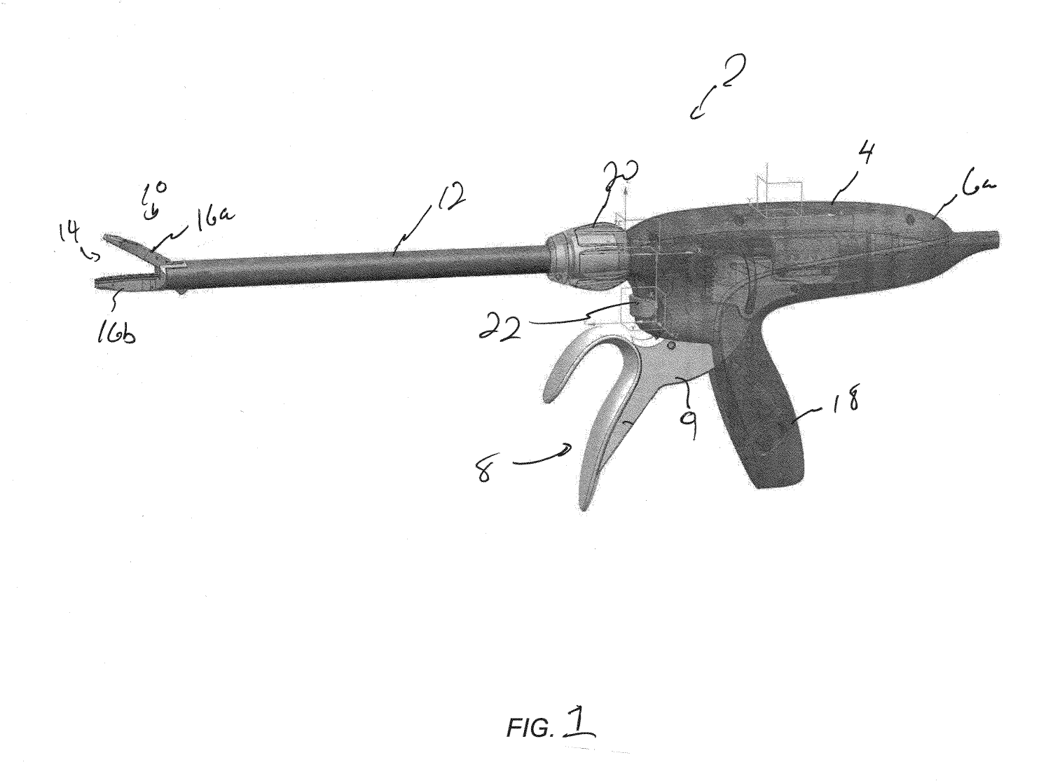

Turning now to the figures, FIG. 1 illustrates a surgical instrument 2 comprising a two stage trigger 8. The two stage trigger 8 is configured to clamp and fire an end effector 12 coupled to the surgical instrument 2 in a single stroke. The surgical instrument 2 comprises a handle assembly 4, a shaft assembly 12, and an end effector 10. The shaft assembly 12 comprises a proximal end and a distal end. The proximal end of the shaft assembly 12 is coupled to the distal end of the handle assembly 4. The end effector 10 is coupled to the distal end of the shaft assembly 12. The handle assembly 4 comprises a pistol grip. The handle assembly 4 comprises a left handle housing shroud 6a and a right handle housing shroud 6b. The two stage trigger 8 comprises a two-stage trigger 9 actuatable towards the pistol grip handle 18. A rotatable shaft knob 20 is configured to rotate the shaft assembly 12 with respect to the handle assembly 4. The handle assembly 4 further comprises an energy button 22 configured to provide electrosurgical energy to one or more electrodes in the end effector 10.

The shaft assembly 12 comprises a jaw actuator, a cutting member actuator 98, and an outer sheath 96. In some embodiments, the outer sheath 96 comprises the jaw actuator. The outer sheath 96 comprises one or more contact electrodes on a distal end configured to interface with the end effector 10. The one or more contact electrodes are operatively coupled to the energy button 22 and an energy source (not shown).

The energy source may be suitable for therapeutic tissue treatment, tissue cauterization/sealing, as well as sub-therapeutic treatment and measurement. The energy button 18 controls the delivery of energy to the electrode. As used throughout this disclosure, a button refers to a switch mechanism for controlling some aspect of a machine or a process. The buttons may be made out of a hard material such as usually plastic or metal. The surface may be formed or shaped to accommodate the human finger or hand, so as to be easily depressed or pushed. Buttons can be most often biased switches, though even many un-biased buttons (due to their physical nature) require a spring to return to their un-pushed state. Terms for the “pushing” of the button, may include press, depress, mash, and punch.

In some embodiments, an end effector 10 is coupled to the distal end of the shaft assembly 12. The end effector 10 comprises a first jaw member 14a and a second jaw member 14b. The first jaw member 14a is pivotably coupled to the second jaw member 14b. The first jaw member 14a is pivotally moveable with respect to the second jaw member 14b to grasp tissue therebetween. In some embodiments, the second jaw member 14b is fixed. In other embodiments, the first jaw member 14a and the second jaw member 14b are pivotally movable. The end effector 10 comprises at least one electrode. The electrode is configured to deliver energy. Energy delivered by the electrode may comprise, for example, radiofrequency (RF) energy, sub-therapeutic RF energy, ultrasonic energy, and/or other suitable forms of energy. In some embodiments, a cutting member (not shown) is receivable within a longitudinal slot defined by the first jaw member 14a and/or the second jaw member 14b. The cutting member is configured to cut tissue grasped between the first jaw member 14a and the second jaw member 14b. In some embodiments, the cutting member comprises an electrode for delivering energy, such as, for example, RF and/or ultrasonic energy.

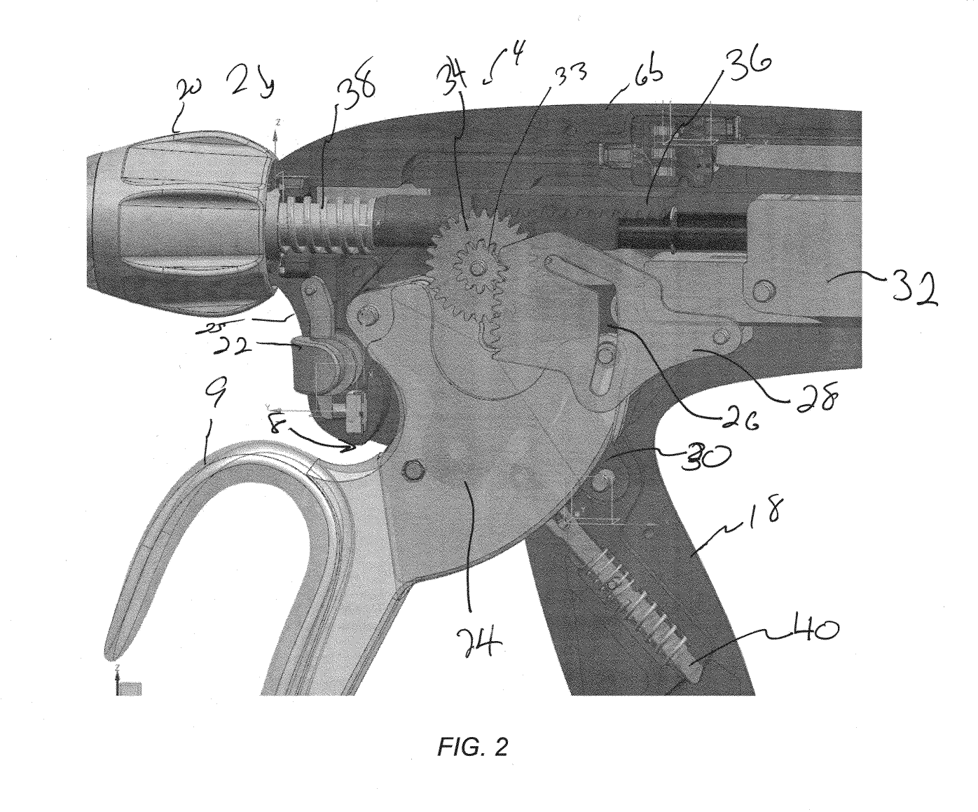

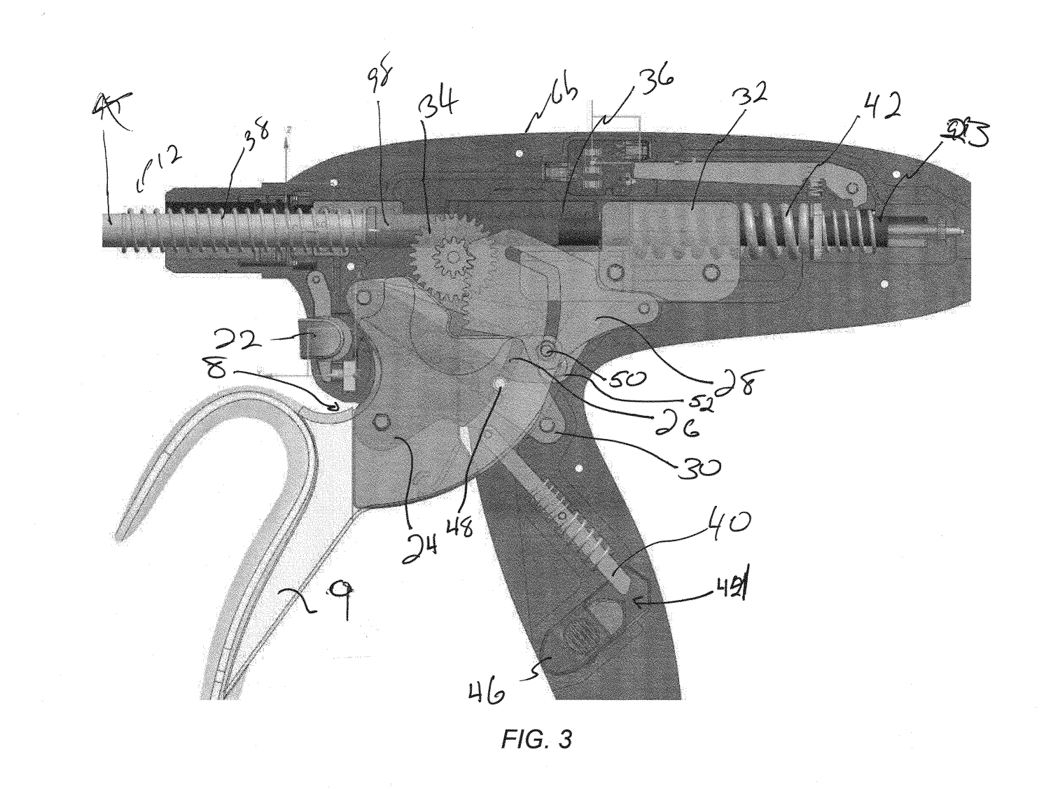

FIGS. 2 and 3 illustrate a side perspective of the surgical instrument 2 with the left handle housing shroud 6a removed. The handle assembly 4 comprises a plurality of components for actuating the surgical instrument 2, such as, for example, mechanism for affecting the closure of the jaws 16a, 16b of the end effector 10, deploying a cutting member within the end effector 10, and/or delivering energy to one or more electrodes coupled to the end effector 10. A two-stage trigger assembly 8 is configured to clamp and fire the end effector 10. The two-stage trigger assembly 8 comprises a two-stage trigger 9. The two-stage trigger 9 is coupled to one or more mechanisms for closing the jaws 16a, 16b of the end effector and deploying a cutting member therein.

In one embodiment, the two-stage trigger assembly 8 comprises a trigger plate 24 coupled to the two-stage trigger 9. Rotation of the two-stage trigger 9 rotates the trigger plate 24 about a rotation point defined by a rotation pin 25. Rotation of the trigger plate 24 to a first rotation causes rotation of a clamp plate 26. The clamp plate 26 is configured to transition the jaws 16a, 16b from an open position to a closed position. For example, in the illustrated embodiment, the clamp plate 26 is coupled to a yoke 32 by a toggle clamp 52. Rotation of the clamp plate 26 drives the yoke 32 proximally. Proximal movement of the yoke 32 compresses a closure spring 42, causing proximal movement of a jaw closure actuator 23. Proximal movement of the jaw closure actuator pivotally moves the first jaw member 16a from an open position to a closed position with respect to the second jaw member 16b.

The two-stage trigger assembly 8 comprises a firing plate 28. Rotation of the trigger plate 24 beyond a predetermined rotation such as, for example, the first rotation, causes rotation of the firing plate 28. Rotation of the firing plate 28 deploys a cutting member within the end effector 10. For example, in the illustrated embodiment, the firing plate 28 comprises a sector gear coupled to a rack 36 and pinion 34. The firing plate 28 comprises a plurality of teeth configured to interface with the pinion 34. Rotation of the firing plate 28 rotates the pinion 34, driving the rack 36 distally. Distal movement of the rack 36 drives a firing actuator 38 distally, causing deployment of the cutting member within the end effector 10.

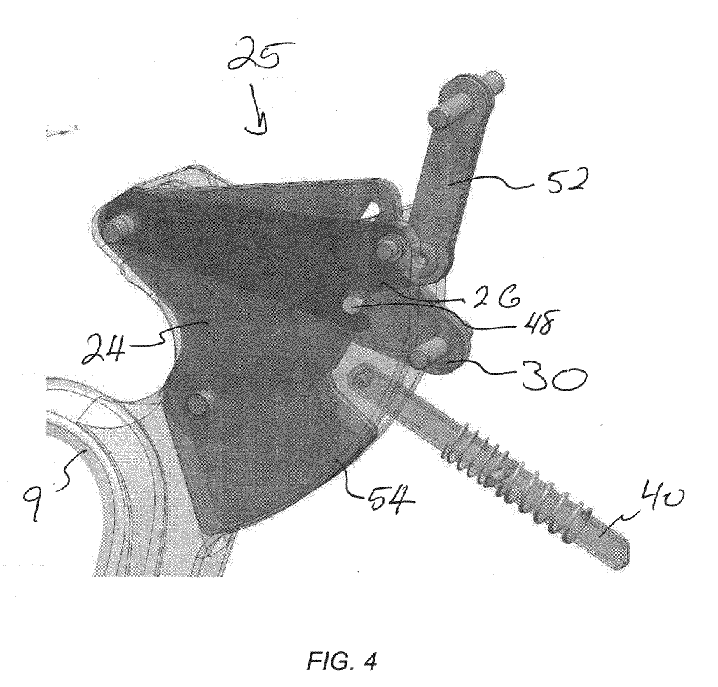

FIG. 4 illustrates one embodiment of a clamp assembly 25 of the two-stage trigger 8. The trigger plate 24 is configured to interface with the clamp plate 26 during rotation of the two-stage trigger 9 from an initial position to a first rotation. The trigger plate 24 is coupled to the clamp plate 26 by a floating pin 48. The floating pin 48 slideably moves within a pin track 56. The pin track 56 may be defined by, for example, a track plate 30 fixedly coupled to at least one of the handle housing shrouds 6a, 6b. Rotation of the trigger plate 24 drives the floating pin 48 within the pin track 56. Movement of the floating pin 48 within a first portion of the pin track 56 engages the clamp plate 26. The pin track 56 comprises a bypass section 58. When the floating pin 48 reaches the bypass section 58 of the pin track 56, the floating pin 48 moves into the bypass section 58 and out of contact with the trigger plate 24 and the clamp plate 26.

The floating pin 48 drives the clamp plate 26 when the trigger plate 24 is rotated from an initial position to a first rotation. Rotation of the clamp plate 26 drives the toggle clamp 52 distally. The toggle clamp 52 is coupled to the yoke 32. Distal movement of the toggle clamp 52 causes distal movement of the yoke 32, which compresses the closure spring 42. Compression of the closure spring 42 causes the jaws 16a, 16b to transition from an open position to a closed position.

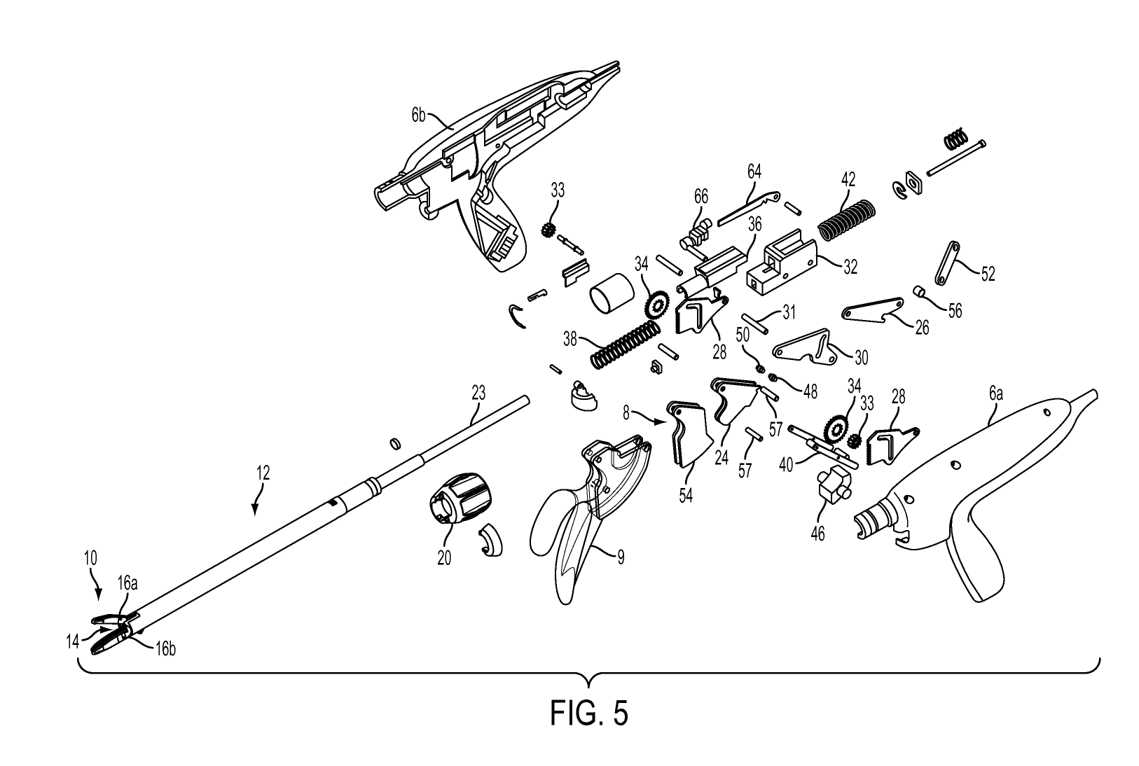

FIG. 5 illustrates an exploded view of the surgical instrument 2 illustrated in FIGS. 1-4. The surgical instrument 2 comprises a left handle housing 6a and a right handle housing 6b. The handle assembly 4 comprises a trigger assembly 8 having a two-stage trigger 9. The two-stage trigger 9 is coupled to a trigger plate 24. The trigger plate 24 is coupled to a clamp plate 26 by a floating pin 48. The floating pin 48 is slideably moveable within a pin track 56 defined by a track plate 30. The trigger plate 24 is further coupled to a firing plate 28 by a firing pin 50. The firing pin 50 is configured to slideably move within a firing pin path 60 defined by the firing plate 28. The trigger plate 24, the clamp plate 26, and the firing plate 28 are coupled to the handle assembly 4 by one or more pivot pins 97. The firing plate 28 is coupled to a rack 36 and pinion 34. Rotation of the firing plate 28 causes the rack 36 to advance distally to deploy a cutting member within a longitudinal slot defined by the jaw assembly 14. The trigger plate 24 and the clamp plate 26 are configured to rotate under the firing plate 28.

In some embodiments, a plunger 40 is configured to provide a physical stop to the two-stage trigger 9 at a first rotation. The plunger 40 is spring biased. The plunger 40 comprises a plunger pin 41 configured to interface with a cam path 68 defined by a cam plate 54. The cam plate 54 is coupled to the trigger plate 24. The cam plate 54 rotates in response to actuation of the two-stage trigger 9. The plunger pin 41 follows the cam path 68 during an actuation stroke of the two-stage trigger 9. The cam path 68 comprises a detent 72. When the plunger pin 41 reaches the detent 72, the plunger 40 springs into place and maintains the two-stage trigger 9 at the first rotation. The trigger 9 may be rotated proximally or distally from the first rotation. The two-stage trigger 9 is rotatable proximally, towards the pistol grip 18, to continue the firing stroke and deploy a cutting member within the end effector 10. The closure trigger is rotatable distally, away from the pistol grip 18, to release the jaws 16a, 16b of the end effector 10.

Referring back to FIG. 3, the surgical instrument 2 is illustrated with the two-stage trigger 9 in an initial position. In the initial position, the two-stage trigger 9 is at a zero rotation position. The trigger plate 24, the clamp plate 26, and the floating pin 48 are in an initial position corresponding to the jaws 16a, 16b being in an open position. The floating pin 48 is located at the bottom of the pin track 56 defined by the track plate 30. The initial position of the two-stage trigger 9 further corresponds to the yoke 32 being in a distal-most position and the closure spring 42 having an initial compression. In some embodiments, the closure spring 42 is uncompressed when the yoke 32 is in a distal-most position. In other embodiments, the closure spring 42 comprises a pre-compressed spring having a first compression when the yoke 32 is in a distal-most position. The rack 36 is located in a proximal-most position.

The trigger plate 24 is coupled to the firing plate 28 by a firing pin 50 located within a firing pin path 60 defined by the firing plate 28. Rotation of the trigger plate 24 drives the firing pin 50 within the firing pin path 60. The firing pin path 60 comprises a non-firing portion 61 and a firing portion 62. When the firing pin 50 moves within the non-firing portion 61, the firing plate 28 remains stationary. As the trigger plate 24 continues to rotate and moves the firing pin 50 into the firing portion 62, movement of the firing pin 50 rotates the firing plate 28 about a pivot point defined by a pivot pin 97b.

In operation, a clinician positions a tissue section for treatment between the first and second jaw members 16a, 16b of the end effector 10. The clinician rotates the two-stage trigger 9 towards the pistol grip handle 18. Rotation of the two-stage trigger 9 rotates the trigger plate 24 and the cam plate 54 about an axis defined by a pivot pin 97a. Rotation of the trigger plate 24 drives the floating pin 48 upward in the pin track 56. The floating pin 48 engages the clamp plate 26 and rotates the clamp plate 26 about the axis defined by the pivot pin 97a. Rotation of the clamp plate 26 drives the toggle clamp 52 proximally, moving the yoke 32 proximally and compressing the closure spring 42.

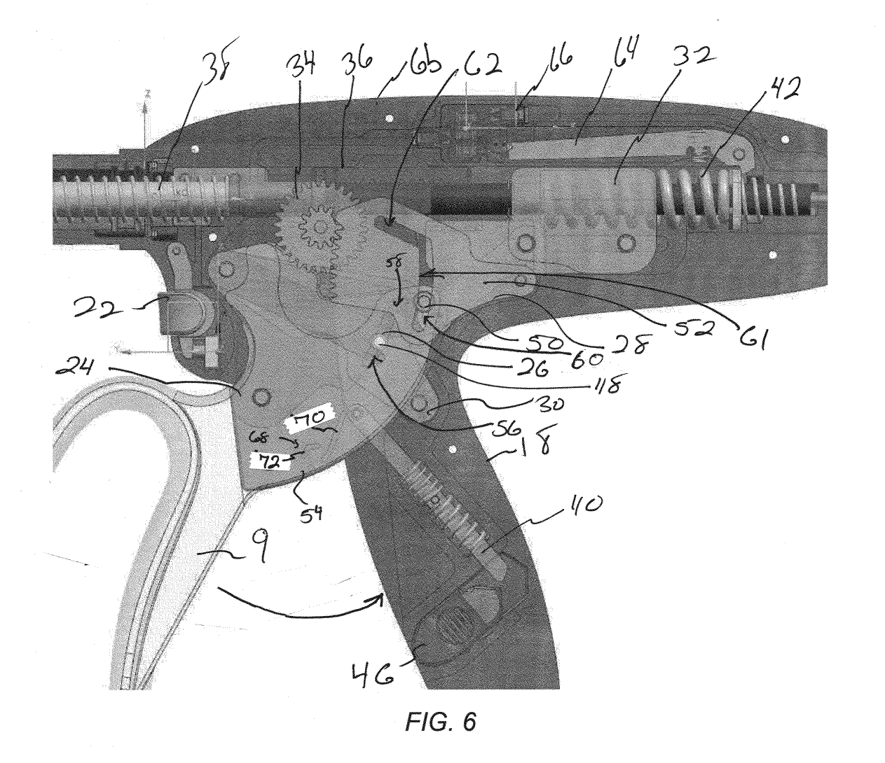

FIG. 6 illustrates the surgical instrument of FIG. 3 with the two-stage trigger 9 in a partially actuated position. The two-stage trigger 9 is illustrated at a rotation of about 7 degrees from the initial, or zero, position illustrated in FIG. 3. As shown in FIG. 6, rotation of the two-stage trigger 9 rotates the trigger plate 24 and drives the floating pin 48 within the pin track 56. The floating pin 48 has been driven upwards within the pin track 56. The floating pin 48 is interfaced with the clamp plate 26 and has partially rotated the clamp plate 26 to drive the toggle clamp 52 proximally. The proximal movement of the toggle clamp 52 causes the yoke 32 to move proximally and compress the closure spring 42. The closure spring 42 is illustrated in a partially compressed position corresponding to a partial closure of the jaw assembly 14.

The rotation of the trigger plate 24 drives the firing pin 50 within the firing pin path 60. The firing pin 50 moves within a first, non-firing portion 61 of the firing pin path 60 when the two-stage trigger 9 is rotated to the first rotation. The firing pin 50 is illustrated within the non-firing portion 61 of the firing pin path 60, as the two-stage trigger 9 has not yet completed a first, or closing, stroke. Rotation of the two-stage trigger 9 further drives the cam plate 54 into contact with the plunger 40. As illustrated in FIG. 6, the plunger 40 contacts the cam path 68 defined by the cam plate 54 when the two-stage trigger 9 is actuated.

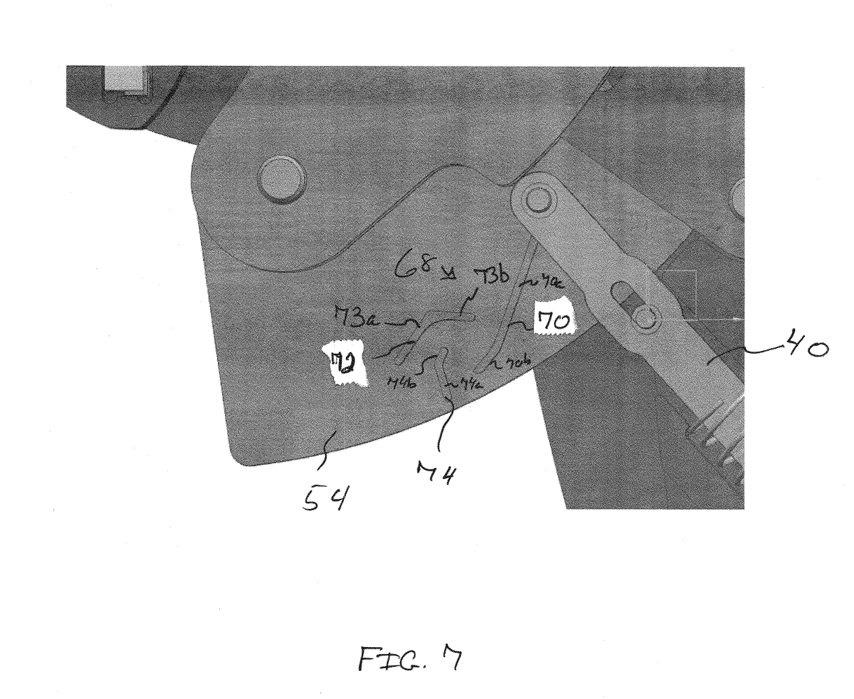

FIG. 7 illustrates the position of a cam plate 54 and a plunger 40 corresponding to the rotation of the closure trigger illustrated in FIG. 6. The cam plate 54 comprises a cam path 68 configured to allow the plunger pin 41 to stop and maintain an intermediate trigger position until an external force is applied. The plunger pin 41 defines a circumference configured to allow passage through the cam path 68. For example, in one embodiment, the plunger pin 41 defines a circumference of 0.063 inches. The angles of the cam path 68 in combination with the plunger 40 spring rates are configured to enable a detent function. As shown in FIG. 7, a plunger pin 41 coupled to the plunger 40 is in contact with an angled portion 70 of the cam path 68. The plunger pin 41 contacts the uppermost end of the angled portion 70. As the cam plate 54 rotates in response to actuation of the two-stage trigger 9, the plunger pin 41 follows the cam path 68 and compresses the plunger 40. In one embodiment, the angled portion 70 of the cam path 68 comprises a first incline 70a and a second incline 70b. A surface of the first incline 70a and a surface of the second incline 70b are offset at a specific angle, such as, for example, 199.4°.

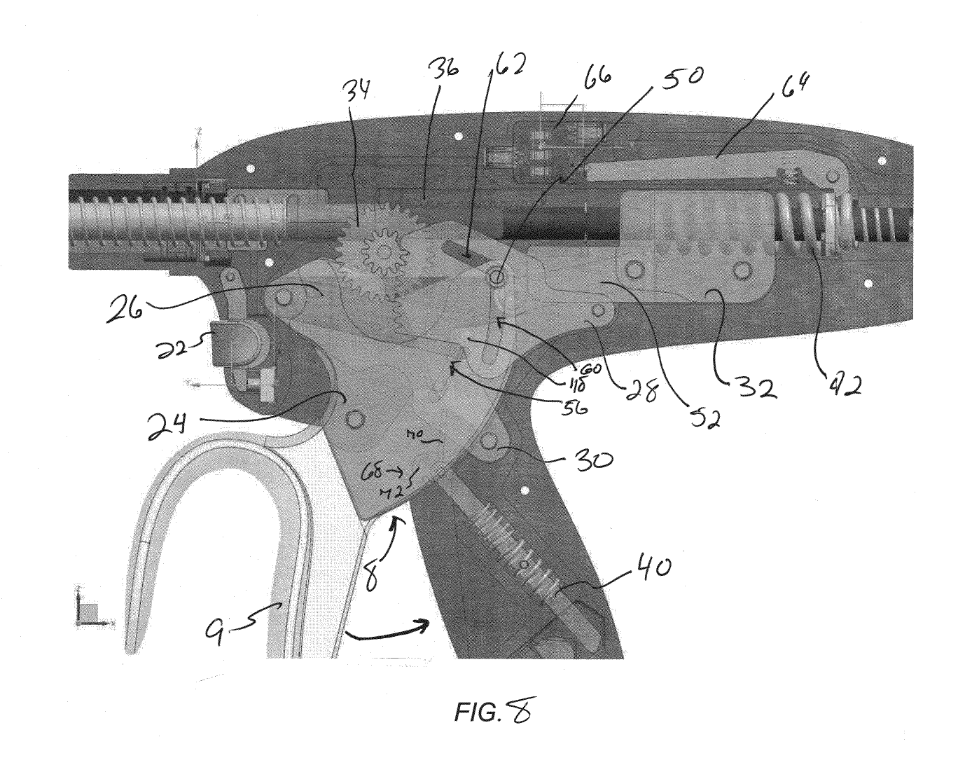

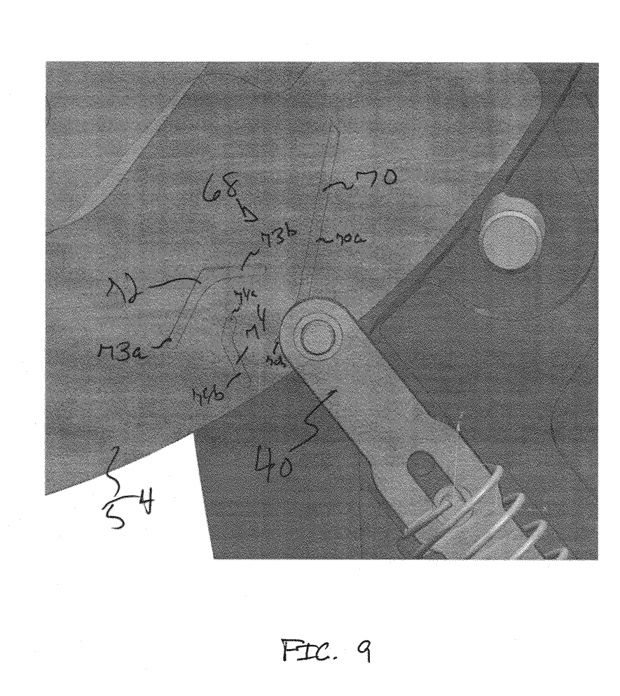

FIG. 8 illustrates the surgical instrument of FIG. 3 having the two-stage trigger 9 further actuated. In the illustrated embodiment, the two-stage trigger 9 comprises a rotation of greater than 7 degrees but less than 24 degrees. The continued rotation of the two-stage trigger 9 to the rotation illustrated in FIG. 8 further advances the trigger plate 24 to drive the floating pin 48 within the floating pin track 56 defined by the track plate 30. The floating pin 48 further drives the clamp plate 26 to drive the toggle clamp 52 and the yoke 32 proximally. In the illustrated embodiment, the yoke 32 has been driven to a proximal-most position and compresses the closure spring 42 to a maximum compression. The position of the yoke 32 and the compression of the closure spring 42 correspond to the jaws 16a, 16b of the end effector 10 being fully closed on a tissue section located therebetween. The trigger plate 24 has rotated the trigger pin 50 to the uppermost section of the non-firing portion 61 of the firing pin path 60. The firing plate 28 remains in the initial position. FIG. 9 illustrates the plunger 40 and the cam plate 54 when the two-stage trigger 9 is at the rotation illustrated in FIG. 8. As shown in FIG. 9, the plunger pin 41 is located at the bottom of the angled portion 70 of the cam path 68. The plunger 40 has been compressed by the angled portion 70 of the cam path 68.

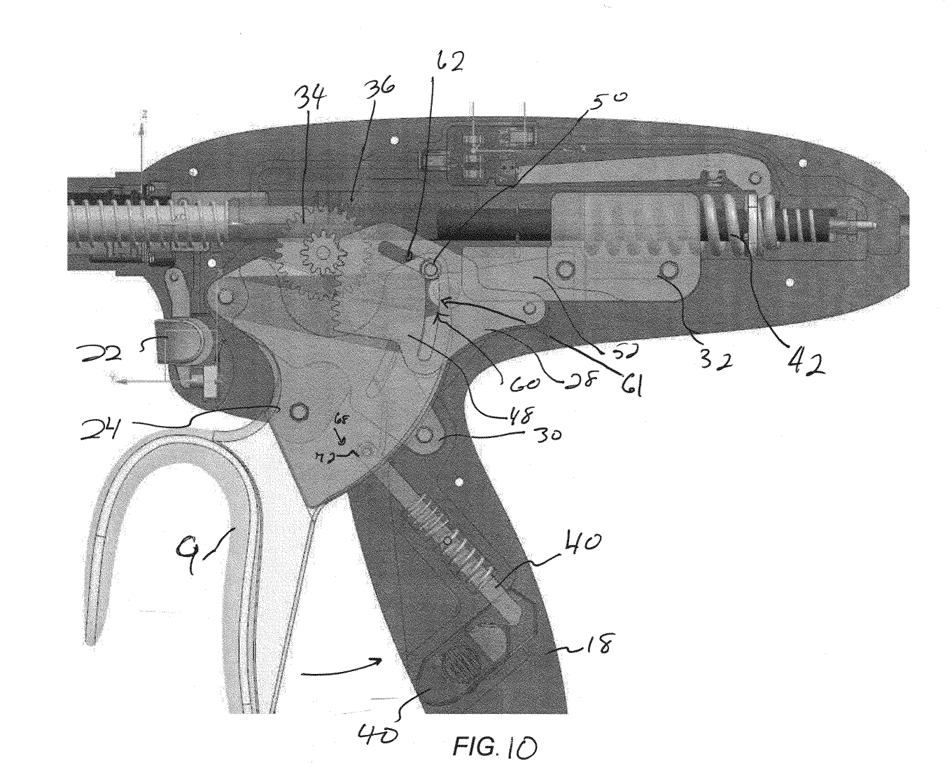

FIG. 10 illustrates the surgical instrument of FIG. 3 having the closure trigger actuated to a first rotation. In the illustrated embodiment, the first rotation corresponds to a rotation of about 24 degrees from the initial, or zero, position. Rotation of the two-stage trigger 9 to the first rotation advances the trigger plate 24 and moves the floating pin 48 into a bypass section 58 of the pin track 56. Movement of the floating pin 48 into the bypass section 58 causes the floating pin 48 to disengage with the trigger plate 24 and the clamp plate 26. Once the floating pin 48 enters the bypass section 58, continued rotation of the trigger plate 24 does not affect the clamp plate 26 and/or the jaws 16a, 16b of the end effector 10. The trigger plate 24 maintains the floating pin 48 in the bypass section 58 when the trigger plate 24 is further actuated. The toggle clamp 52 and the yoke 32 are maintained in a proximal-most position. In some embodiments, the yoke 32 moves slightly distally when the floating pin 48 enters the bypass section, causing the jaws 16a, 16b to reduce a force applied to a tissue section located therebetween. The clamp plate 26 rotates almost to center, but does not cross over center, allowing the clamp plate 26 to return to a starting position based on a force exerted by the closure spring 42 on the yoke 32 when the trigger 9 is released.

The rotation of the two-stage trigger 9 to the first rotation drives the trigger pin 50 to a transition position within the trigger pin track 60. Continued rotation of the two-stage trigger 9 drives the trigger pin 50 into the rotation section 62 of the trigger pin track 60. As can be seen in FIG. 10, the trigger plate 24 and the clamp plate 26 rotate beneath the firing plate 28 when rotated by actuation of the two-stage trigger 9. Rotation of the trigger 9 to the first rotation causes the plunger 40 to engage with a detent 72 of the cam path 68.

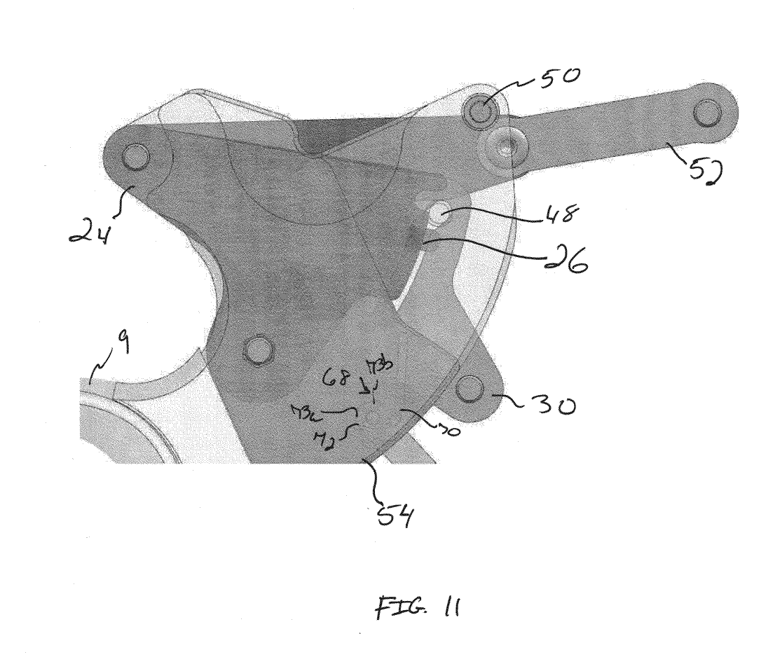

FIG. 11 illustrates the plunger 40 and the cam plate 54 when the trigger 9 has been rotated to the first rotation, as illustrated in FIG. 10. When the trigger 9 is rotated from a position less than the first rotation (for example, the position illustrated in FIG. 9) to the first rotation, the cam plate 54 rotates such that the plunger pin 41 disengages from the angled portion 70 of the cam path 68 and springs into contact with the detent 72. A vertical stop 74 prevents over rotation of the trigger 9 before the plunger 40 springs into contact with the detent 72. In some embodiments, the vertical stop 74 comprises a first portion 74a and a second portion 74b. The first portion 74a and the second portion 74b are offset by a predetermined angle, such as, for example, 220.1°. The angle portion 70 of the cam path 68 and the vertical stop 74 are separated by a gap, such as, for example, a gap of 0.074 inches.

In some embodiments, the plunger 40 provides tactile and/or audible feedback to a clinician to indicate that the plunger pin 41 has moved into contact with the detent 72. The plunger 40 maintains the cam plate 54 and the trigger 9 at the first rotation until a predetermined force is applied to disengage the plunger 40 from the detent 72. In operation, a clinician may continue rotating the trigger 9 proximally to deploy the cutting member within the end effector 10 or may rotate the trigger 9 distally to open the jaws and return the surgical instrument 2 to the initial position illustrated in FIG. 3. The detent 72 comprises arms 73a, 73b that define the force necessary to deploy the cutting member and/or return the jaws 16a, 16b to an open position. The angle of the arms 73a, 73b determines the force necessary to disengage the plunger pin 41 from the detent 72 in a proximal or distal direction. In some embodiments, the first arm 73a is offset from the second arm 73b by an angle of, for example, 240.0°. Those skilled in the art will recognize that any suitable angle may be used. In some embodiments, the first arm 73a and the vertical stop 74 define a first spacing therebetween and the second arm 73b and the vertical stop 74 define a second spacing therebetween. The first spacing may comprise, for example, a spacing of about 0.074 inches. The second spacing may comprise, for example, a spacing of about 0.085 inches. The second arm 73b and the angled portion 70 of the cam path 68 define a spacing therebetween, such as, for example, 0.080 inches to allow the spring pin 41 to pass unimpeded during a return stroke.

Rotation of the two-stage trigger 9 proximally deploys a cutting member within the end effector 10. Rotation of the trigger 9 beyond the first rotation disengages the plunger 40 from the detent 72. The force required to continue rotation of the two-stage trigger 9 to deploy the cutting member is defined by a proximal arm 73a of the detent 72 of the cam path 68. The trigger 9 rotates the trigger plate 24 to slideably move the trigger pin 50 within the rotation portion 62 of the trigger pin path 60. Movement of the trigger pin 50 within the rotation portion 62 causes the firing plate 28 to rotate about an axis defined by the pivot pin 97b. Rotational movement of the firing plate 28 causes the pinion 34 to rotate to drive the rack 36 distally. Distal movement of the rack 36 deploys the cutting member distally within the end effector 10.

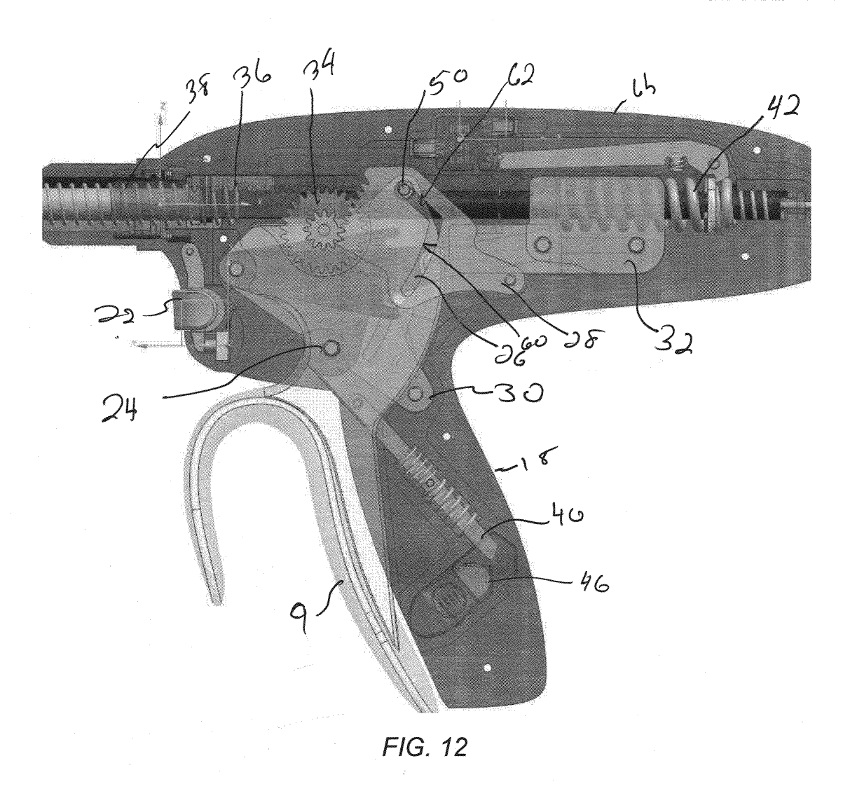

FIG. 12 illustrates the surgical instrument of FIG. 3 in a fully fired position. The two-stage trigger 9 of the two stage trigger assembly 8 has been fully rotated proximally. In the illustrated embodiment, full rotation of the two-stage trigger 9 corresponds to a rotation of about 42 degrees from the initial position illustrated in FIG. 3. As shown in FIG. 12, the trigger plate 24 has been rotated under the firing plate 28. The firing pin 50 is located at the top of the firing pin path 60. Movement of the firing pin 50 within the rotation section 62 of the firing pin path 60 rotates the firing plate 28. Rotation of the firing plate 28 rotates the pinion 34 and advances the rack 36 distally. Distal movement of the rack 36 deploys the cutting member within the end effector 10. After the cutting member has been fully deployed, the clinician may release the two-stage trigger 9 to return the surgical instrument 2 to the initial position illustrated in FIG. 3. FIG. 12 illustrates the surgical instrument 2 in a fully fired position. In some embodiments, the mechanical advantage of the trigger 9 increases as the rotation of the trigger 9 increases.

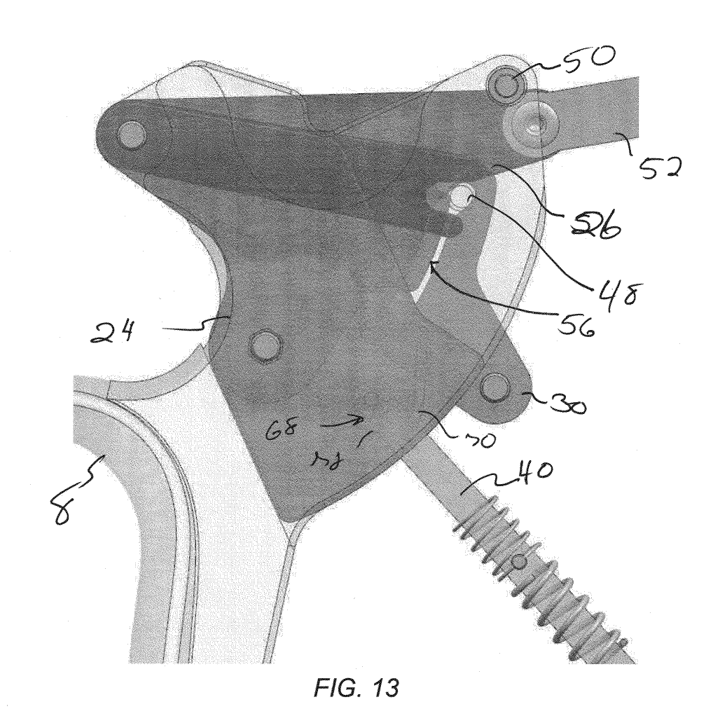

In some embodiments, the plunger 40 is configured to bypass the cam path 68 during a return stroke. FIG. 13 illustrates a position of the plunger 40 with respect to the cam path 68 during a return stroke of the two-stage trigger assembly 8. The plunger 40 is spring biased, causing the plunger pin 41 to bypass the detent 72 of the cam path 68 during a return stroke. FIG. 13 illustrates the two-stage trigger 9 in a partially returned position. By bypassing the detent 72 of the cam path 68, the two-stage trigger assembly 8 provides a smooth return from the fully fired position illustrated in FIG. 12 to the initial position illustrated in FIG. 3. In some embodiments, the plunger pin 41 contacts the angled portion 70 of the cam path 68 to provide a dampening force to the return stroke of the two-stage trigger 9. The force of one or more springs, such as, for example, a firing spring 38 and/or the closure spring 52, automatically returns the trigger 9 to an initial position when the trigger 9 is released. In some embodiments, the clinician may manually rotate the trigger 9 to the initial position after firing.

In some embodiments, one or more elements of the two-stage trigger assembly 8 may comprise a lubricated and/or low friction material. For example, in some embodiments, one or more of the trigger plate 24, clamping plate 26, firing plate 28, track plat 30, cam plate 54 may comprise a lubricated and/or low friction material. In some embodiments, one or more of the pins, such as, for example, the plunger pin 41, the floating pin 48, and/or the firing pin 50 may comprise a lubricated and/or low friction material. Suitable low-friction materials for one or more plates and/or one or more pins comprise, for example, spinodal bronze, Nitronic 60, Cobalt 6B, Waukesha 88, Stellite, and/or Alloy 25. In some embodiments, one or more plates and/or one or more pins may comprise a lubricant coating, such as, for example, Nitrided, Titanium Nitride (TiN), Aluminum Titanium Nitride (AITiN), and/or Malcomized coatings.

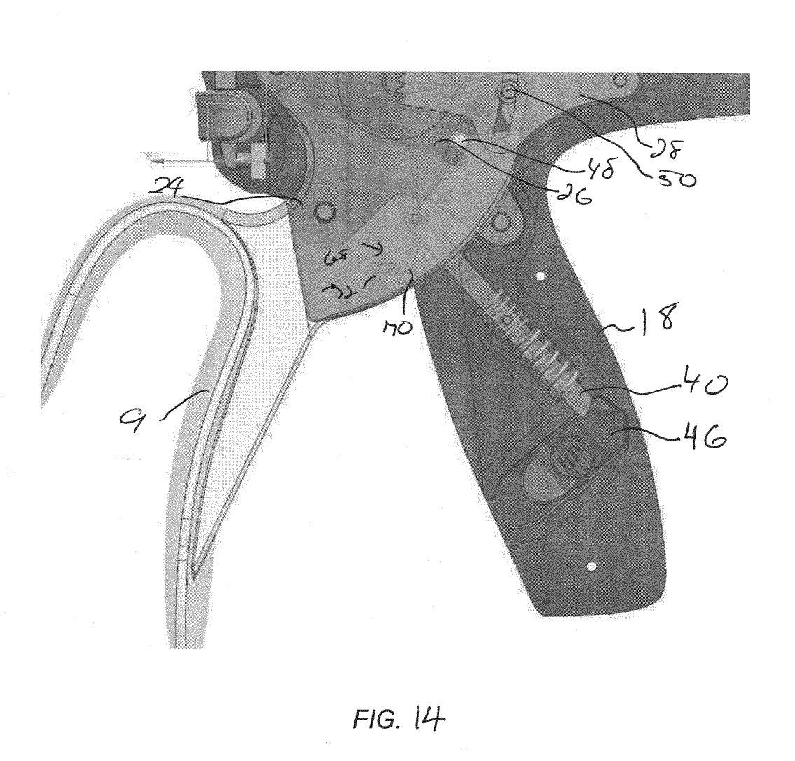

In some embodiments, the surgical instrument 2 comprises a two-stage bypass that allows the trigger assembly 8 to operate as a single stroke clamp and cut trigger. FIG. 14 illustrates one embodiment of the surgical instrument 2 comprising a two-stage bypass switch 46. The two-stage bypass switch 46 comprises a toggle switch that is slideable within the pistol-grip handle 18 of the surgical instrument 2. When the two-stage bypass switch 46 is toggled to a bypass position, as illustrated in FIG. 14, the two-stage bypass switch 46 raises the plunger 40. By raising the plunger 40, the two-stage bypass switch 46 causes the plunger 40 to bypass the cam path 64 during a clamping and firing stroke. Bypassing the cam path 64 prevents the plunger from interfacing with the detent 72 and allows a single, uninterrupted trigger pull to clamp and fire the end effector 10 coupled to the surgical instrument 2.

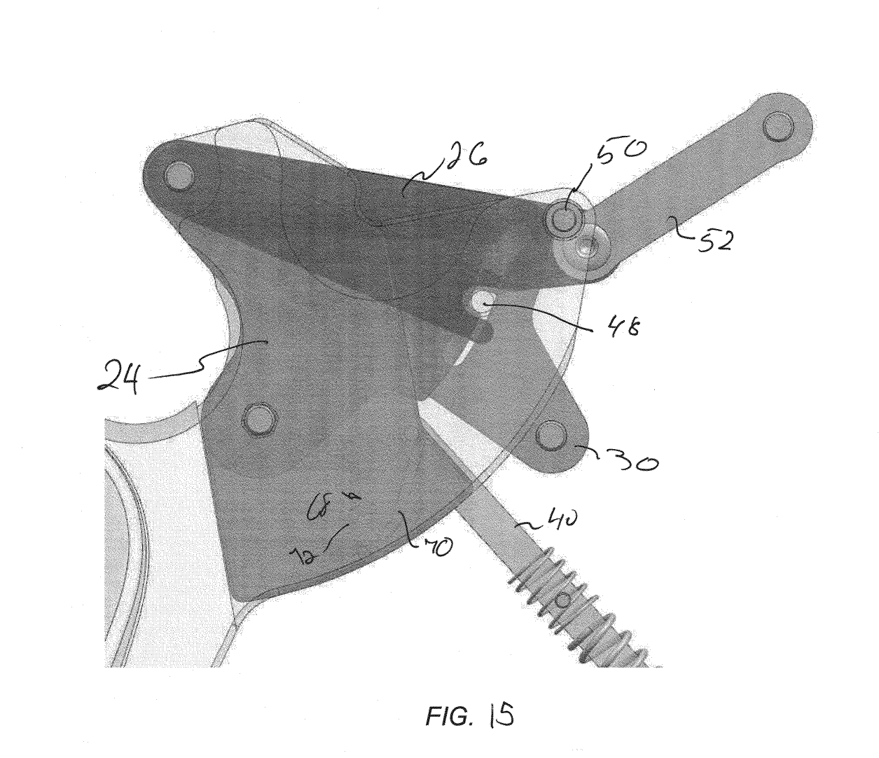

FIG. 15 illustrates the plunger 40 in a bypass position. As illustrated in FIG. 15, the two-stage bypass switch 46 raises the plunger 40 above the upper-most section of the angled portion 70 of the cam path 64. When the two-stage trigger 9 is actuated, the plunger 40 bypasses the angled portion 70 of the cam path 68 and the detent 72. When the cam path 68 is bypassed, the two-stage trigger assembly 8 operates as a single-stage trigger and allows a clinician to clamp and fire the surgical instrument 2 in a single motion.

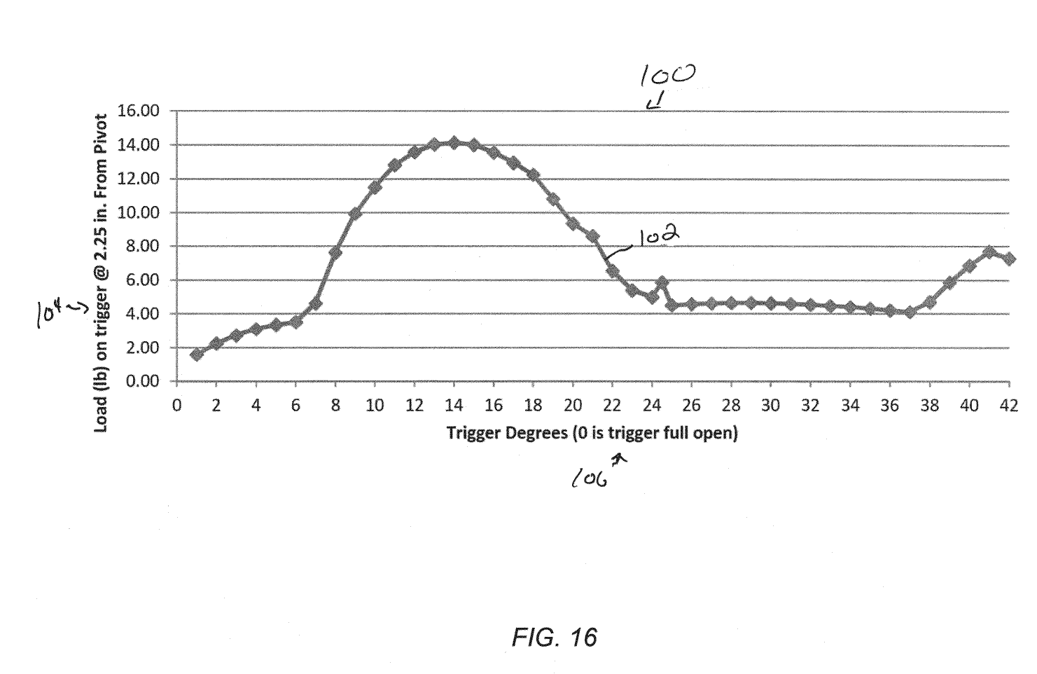

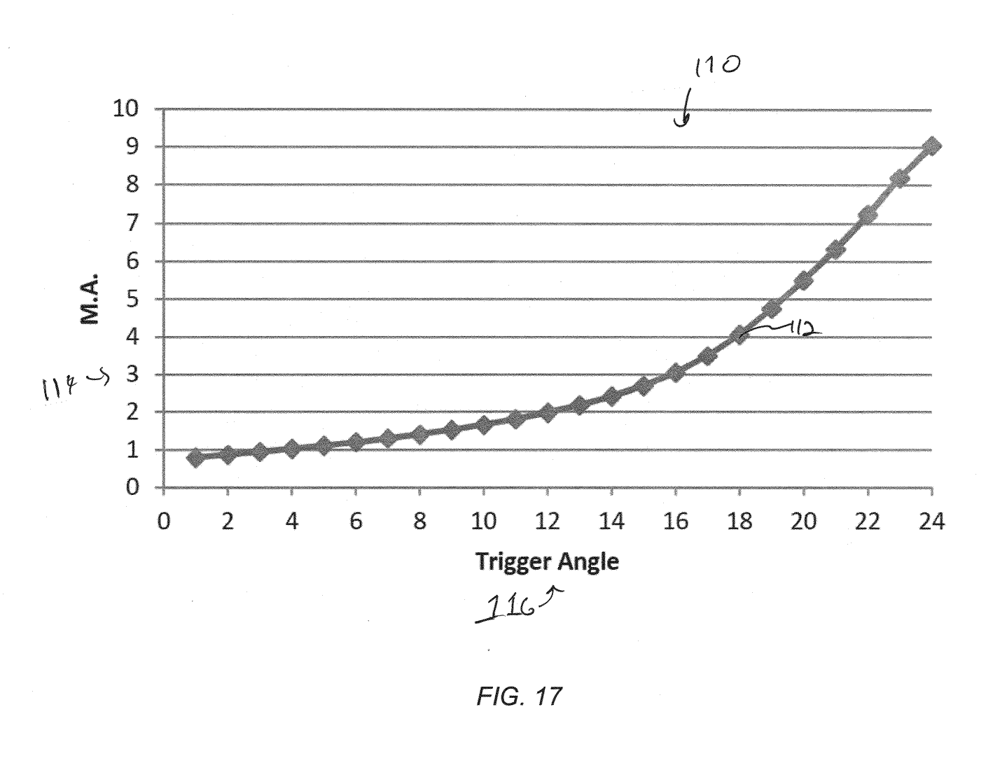

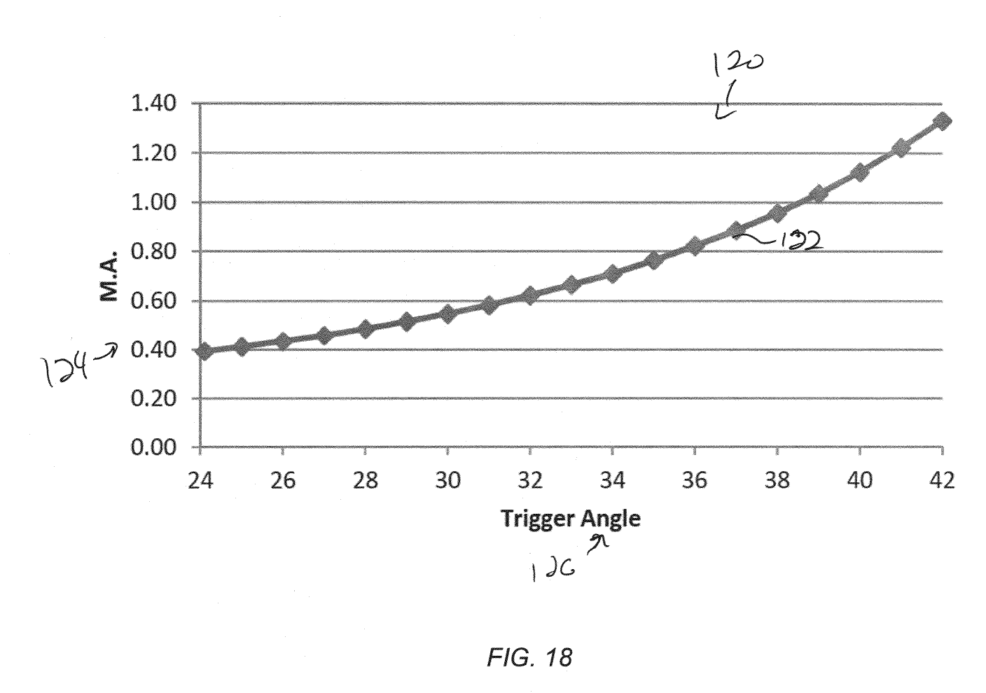

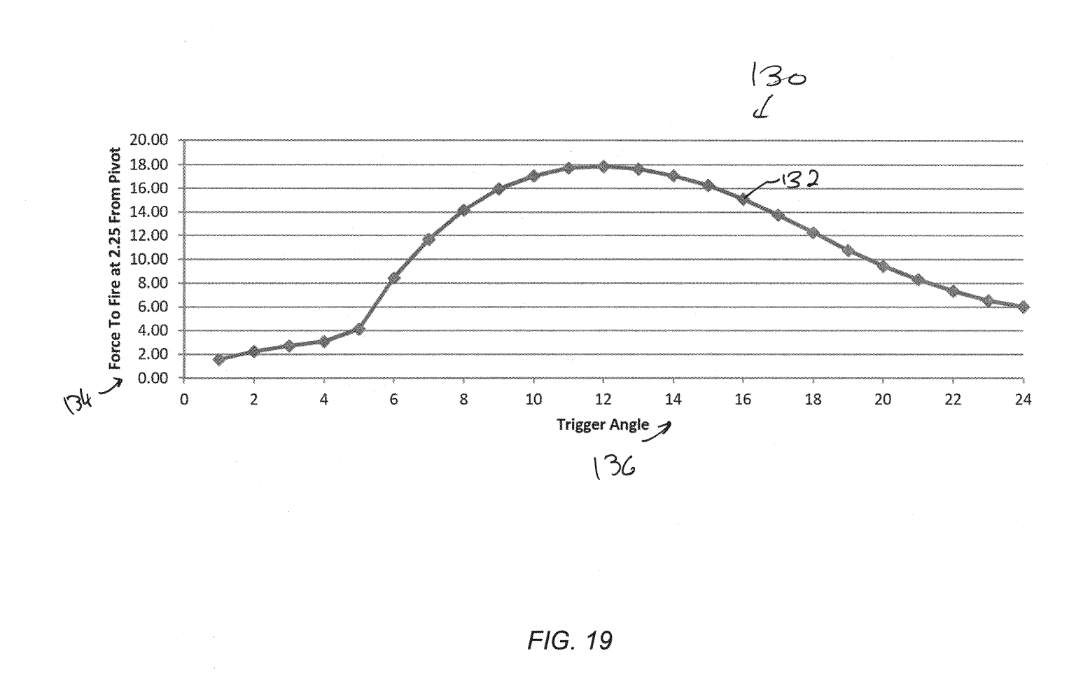

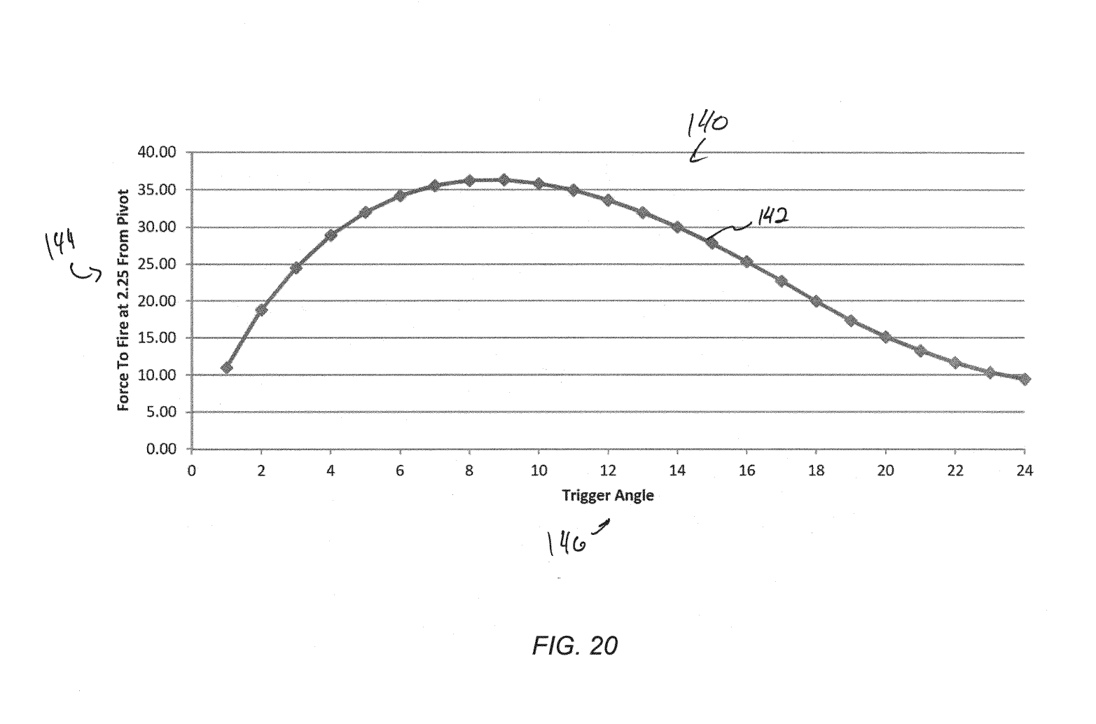

FIG. 16 is a chart illustrating the force to fire the surgical instrument 2 illustrated in FIGS. 1-15. As shown in FIG. 16, the force to fire 102 increases as the two-stage trigger 9 is rotated to a partial rotation. The force to fire 102 begins to decrease when the jaws 16a, 16b are partially closed. The spike at about 24 degrees corresponds to the plunger 40 interfacing with the detent 72 of the cam path 68 and the force required to overcome the arm 73a of the detent 72 to continue rotation of the two-stage trigger 9. FIG. 17 is a graph illustrates the mechanical advantage of the two-stage trigger assembly 8 from an initial position to a first rotation corresponding to a clamping stroke of the two-stage trigger 9. As shown in FIG. 17, the mechanical advantage of the two-stage trigger 9 increases as the two-stage trigger 9 is rotated towards the pistol-grip handle 18. FIG. 18 illustrates the mechanical advantage of the two-stage trigger 9 from the first rotation to a fully fired position. As shown in FIG. 18, the mechanical advantage of the two-stage trigger 9 increases as the two-stage trigger 9 is rotated from the first position to a fully fired position. FIG. 19 is a graph illustrating one embodiment of a clamp load at a jaw assembly of an end effector when the jaw assembly is partially open. FIG. 20 illustrates one embodiment of a force to fire 140 for a clamp stroke 142 of a surgical instrument 2 comprising a two-stage trigger 8.

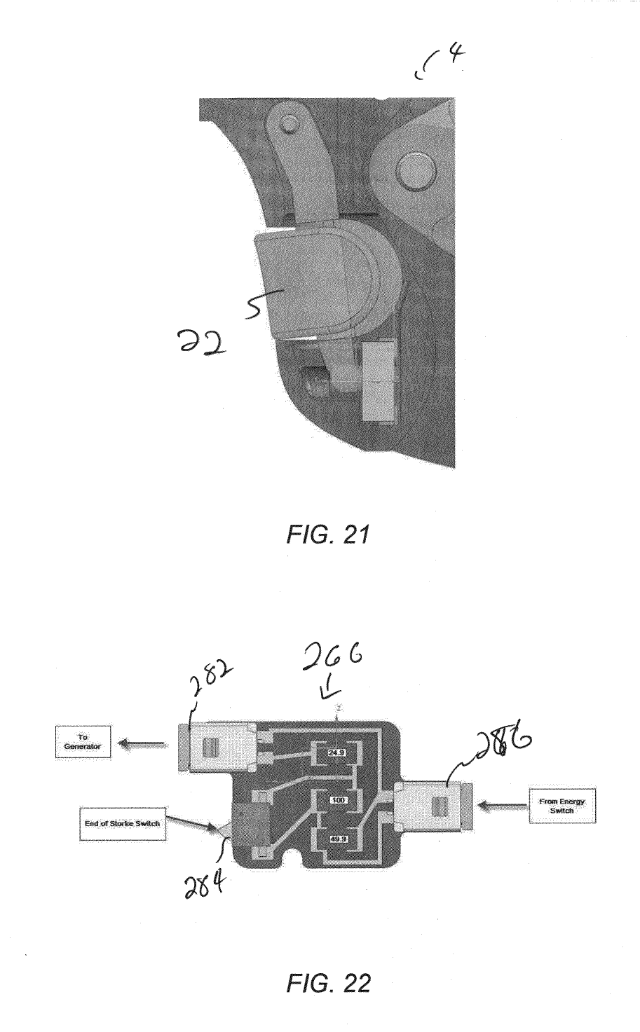

FIG. 21 illustrates one embodiment of an energy delivery button 22 coupled to the handle portion 4 of the surgical instrument 2. The energy delivery button 22 is configured to provide energy to one or more electrodes coupled to the end effector 10. In some embodiments, a clinician may actuate the energy delivery button 22 after clamping tissue within the end effector 10 and prior to deploying the cutting member within the end effector 10. For example, in some embodiments, when the plunger 40 interfaces with the detent 72 to indicate a fully clamped position of the end effector 10, the clinician actuates the energy delivery button 22 to provide therapeutic, sub-therapeutic, ultrasonic, and/or other energy to a tissue section clamped between the jaws 16a, 16b. After applying energy to the tissue section, the clinician actuates the closure trigger 8 to deploy the cutting member within the end effector 10 and cut the tissue section grasped between the first and second jaw members 16a, 16b.

FIG. 22 illustrates one embodiment of an energy circuit 266. The energy circuit 266 may be formed integrally with the handle assembly 4 of the surgical instrument 2. The energy circuit 266 comprises a generator connection 282, an end of stroke switch 284, and an energy switch connection 286. The generator connection 282 is configured to couple the energy circuit 266 to a generator. The generator may be located externally to the surgical instrument 2 and/or may be formed integrally with the handle assembly 4. The energy switch connection 286 couples the energy circuit 266 to the energy delivery button 22 of the surgical instrument 4. The end of stroke switch 284 is configured to prevent delivery of energy to the electrodes of the end effector 10 when the end effector 10 is not in a closed position.

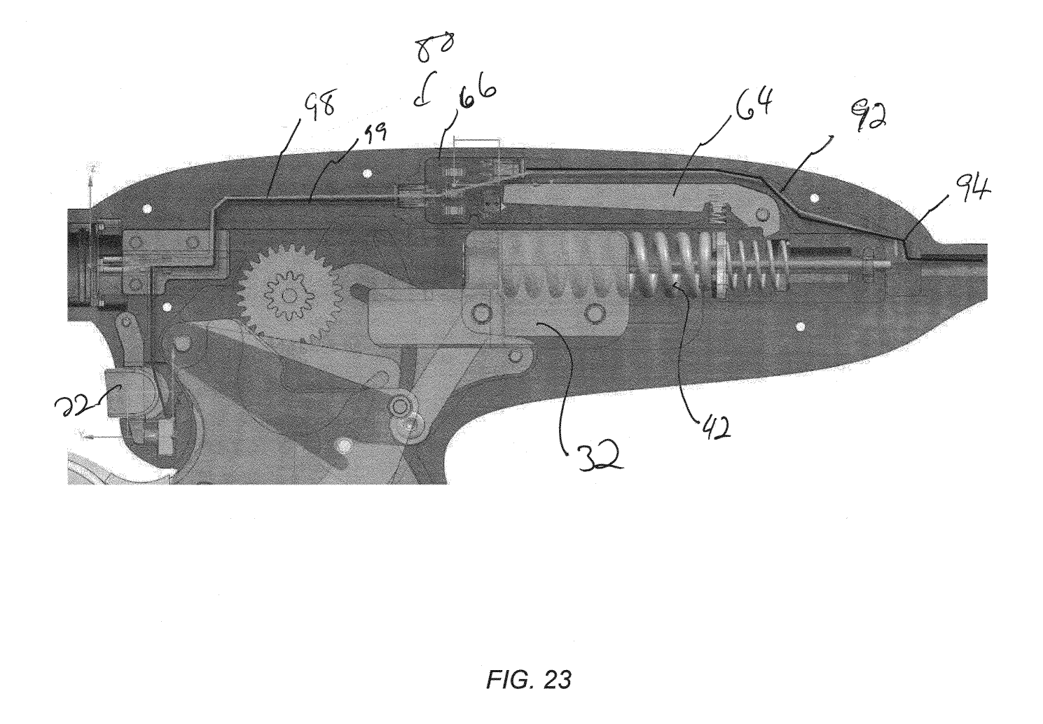

FIG. 23 illustrates an energy system 88 of a surgical instrument, such as, for example, the surgical instrument 2 illustrated in FIGS. 1-15. The energy system 88 comprises an energy circuit 66, such as, for example, the energy circuit 266 illustrated in FIG. 22. A lockout bar 64 is configured to pivotably interface with the end of stroke switch 86 of the energy circuit 66. Proximal movement of the yoke 32 pivots the lockout bar 64 and actuates the end of stroke switch 84 of the energy circuit 66. When the yoke 32 is in an initial position, as illustrated in FIG. 23, the end of stroke switch 84 is in an open position. An open end of stroke switch 84 prevents delivery of energy to the end effector 10 when the energy delivery button 22 is actuated. When the end of stroke switch 66 is actuated, the energy circuit 66 is configured to provide energy to the electrodes of the end effector 10 when the energy button 22 is actuated. The energy circuit 66 is coupled to the generator (not shown) by a generator source wire 92 and a generator return wire 94. A handle source wire 98 and a handle return wire 99 coupled the energy circuit 66 to the energy delivery button 22 and the electrodes of the end effector 10.

While the examples herein are described mainly in the context of electrosurgical instruments, it should be understood that the teachings herein may be readily applied to a variety of other types of medical instruments. By way of example only, the teachings herein may be readily applied to tissue graspers, tissue retrieval pouch deploying instruments, surgical staplers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those of ordinary skill in the art.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The disclosed embodiments have application in conventional endoscopic and open surgical instrumentation as well as application in robotic-assisted surgery.

Embodiments of the devices disclosed herein can be designed to be disposed of after a single use, or they can be designed to be used multiple times. Embodiments may, in either or both cases, be reconditioned for reuse after at least one use. Reconditioning may include any combination of the steps of disassembly of the device, followed by cleaning or replacement of particular pieces, and subsequent reassembly. In particular, embodiments of the device may be disassembled, and any number of the particular pieces or parts of the device may be selectively replaced or removed in any combination. Upon cleaning and/or replacement of particular parts, embodiments of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, embodiments described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta or gamma radiation, ethylene oxide, or steam.

It is worthy to note that any reference to “one aspect,” “an aspect,” “one embodiment,” or “an embodiment” means that a particular feature, structure, or characteristic described in connection with the aspect is included in at least one aspect. Thus, appearances of the phrases “in one aspect,” “in an aspect,” “in one embodiment,” or “in an embodiment” in various places throughout the specification are not necessarily all referring to the same aspect.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken as limiting.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely examples and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

Some aspects may be described using the expression “coupled” and “connected” along with their derivatives. It should be understood that these terms are not intended as synonyms for each other. For example, some aspects may be described using the term “connected” to indicate that two or more elements are in direct physical or electrical contact with each other. In another example, some aspects may be described using the term “coupled” to indicate that two or more elements are in direct physical or electrical contact. The term “coupled,” however, also may mean that two or more elements are not in direct contact with each other, but yet still co-operate or interact with each other.

In some instances, one or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

In summary, numerous benefits have been described which result from employing the concepts described herein. The foregoing description of the one or more embodiments has been presented for purposes of illustration and description. It is not intended to be exhaustive or limiting to the precise form disclosed. Modifications or variations are possible in light of the above teachings. The one or more embodiments were chosen and described in order to illustrate principles and practical application to thereby enable one of ordinary skill in the art to utilize the various embodiments and with various modifications as are suited to the particular use contemplated. It is intended that the claims submitted herewith define the overall scope.

Various aspects of the subject matter described herein are set out in the following numbered clauses:

1. A surgical instrument comprising: a handle assembly comprising: a closure trigger; a push plate coupled to the closure trigger, wherein actuation of the closure trigger rotates the push plate; a clamp plate coupled to the push plate, wherein actuation of the closure trigger to a first rotation rotates the clamp plate; a firing plate coupled to the push plate, wherein actuation of the closure trigger between the first rotation and a second rotation rotates the firing plate; a shaft assembly comprising a proximal end and a distal end, wherein the shaft assembly is coupled to the handle assembly at the proximal end; and an end effector coupled to the distal end of the shaft assembly, the end effector comprising: a jaw assembly having a proximal end and a distal end, the jaw assembly comprising: a first jaw member; and a second jaw member, wherein the first and second jaw members define a longitudinal slot, wherein rotation of the clamp plate pivotally moves the first jaw member from an open position to a closed position relative to the second jaw member; and a cutting member deployable within the longitudinal slot, wherein rotation of the firing plate advances the cutting member distally within the longitudinal slot.

2. The surgical instrument of clause 1, comprising a floating pin coupled to the clamp plate, wherein the floating pin is configured to move within a floating pin path comprising a bypass section, wherein rotation of the closure trigger causes the push plate to move the floating pin within the floating pin path, and wherein the floating pin enters the bypass section and disengages from the push plate to disconnect the push plate from the clamp plate.

3. The surgical instrument of clause 2, wherein the push plate is configured to maintain the floating pin in the bypass section during rotation of the closure trigger from the first rotation to the second rotation.

4. The surgical instrument of clause 3, wherein the clamp plate comprises a toggle clamp.

5. The surgical instrument of clause 4, comprising: a yoke coupled to the toggle clamp, wherein the toggle clamp is configured to drive to the yoke plate proximally, wherein proximal movement of the yoke transitions the jaws from an open position to a closed position; and a closure spring, wherein the closure spring is compressed by proximal movement of the yoke.

6. The surgical instrument of clause 1, comprising a trigger pin coupled to the push plate, wherein the trigger pin is configured to move within a trigger pin path defined by the firing plate, wherein the trigger pin path comprises a first section and a second section, wherein rotation of the closure trigger slideably moves the trigger pin within the trigger pin path, wherein the trigger pin moves freely within the first section of the trigger pin path and wherein the trigger pin rotates the firing plate within the second section.

7. The surgical instrument of clause 1, wherein at least one the push plate, the clamp plate, or the firing plate comprises a low-friction material.

8. The surgical instrument of clause 7, comprising: a rack and pinion coupled to the firing plate, wherein the rack and pinion is configured to pivotally move the first jaw member from an open position to a closed position relative to the second jaw.

9. The surgical instrument of clause 6, wherein a mechanical advantage of the closure trigger changes as the trigger pin traverses the trigger pin path.

10. The surgical instrument of clause 1, comprising: a cam shaft plate coupled to the closure trigger, wherein the cam shaft plate defines a cam path having a detent; and a plunger comprising a plunger pin configured to interface with the cam path, wherein proximal actuation of the closure trigger rotates the cam shaft plate into contact with the plunger, wherein the plunger pin follows the cam path to interface with the detent, and wherein the detent maintains the cam shaft plate and the closure trigger at a first rotation corresponding to the first and second jaw members in a closed position.

11. The surgical instrument of clause 10, wherein the detent comprises a first arm and a second arm, and wherein the first arm defines a force required to continue rotation of the closure trigger to fire the cutting member, and wherein the second arm defines a force required to rotate the closure trigger distally to open the jaws.

12. The surgical instrument of clause 10, comprising a toggle switch coupled to the plunger, wherein the toggle switch comprises a first position and a second position, wherein when the toggle pin is in the first position the plunger pin is configured to interface with the cam path, and wherein when the toggle pin is in the second position the plunger pin is configured to bypass the cam path.

13. A surgical instrument comprising: a handle assembly comprising: a closure trigger; a cam shaft plate coupled to the closure trigger, wherein the cam shaft plate defines a cam path having a detent; a plunger comprising a plunger pin configured to interface with the cam path, wherein proximal actuation of the closure trigger rotates the cam shaft plate into contact with the plunger pin, wherein the plunger pin follows the cam path to interface with the detent, and wherein the detent maintains the cam shaft plate and the closure trigger at a first rotation; a shaft assembly comprising a proximal end and a distal end, wherein the shaft assembly is coupled to the handle assembly at the proximal end; and an end effector coupled to the distal end of the shaft assembly, the end effector comprising: a jaw assembly having a proximal end and a distal end, the jaw assembly comprising: a first jaw member; and a second jaw member, wherein the first and second jaw members define a longitudinal slot, wherein rotation of the clamp plate pivotally moves the first jaw member from an open position to a closed position relative to the second jaw member; and a cutting member deployable within the longitudinal slot, wherein rotation of the firing plate advances the cutting member distally within the longitudinal slot.

14. The surgical instrument of clause 13, wherein the detent comprises a first arm and a second arm, and wherein the first arm sets a force required to continue rotation of the closure trigger to fire the cutting member, and wherein the second arm sets a force required to rotate the closure trigger distally to open the jaws.

15. The surgical instrument of clause 13, comprising a toggle switch coupled to the plunger, wherein the toggle switch comprises a first position and a second position, wherein when the toggle pin is in the first position the plunger is configured to interface with the cam path, and wherein when the toggle pin is in the second position the plunger is configured to bypass the cam path.

16. The surgical instrument of clause 13, comprising: a push plate coupled to the closure trigger, wherein actuation of the closure trigger rotates the push plate; a clamp plate coupled to the push plate, wherein actuation of the closure trigger to a first rotation rotates the clamp plate; and a firing plate coupled to the push plate, wherein actuation of the closure trigger between the first rotation and a second rotation rotates the firing plate.

17. The surgical instrument of clause 16, comprising a floating pin coupled to the clamp plate, wherein the floating pin is configured to move within a floating pin path comprising a bypass section, wherein rotation of the closure trigger causes the push plate to move the floating pin within the floating pin path, and wherein the floating pin enters the bypass section and disengages from the push plate to disconnect the push plate from the clamp plate.

18. The surgical instrument of clause 17, wherein the push plate is configured to maintain the floating pin in the bypass section during rotation of the closure trigger from the first rotation to the second rotation.

19. The surgical instrument of clause 17, wherein at least one of the plunger pin or the floating pin comprises a low-friction material.

20. A surgical instrument comprising: a handle assembly comprising: a closure trigger; a push plate coupled to the closure trigger, wherein actuation of the closure trigger rotates the push plate; a clamp plate coupled to the push plate, wherein actuation of the closure trigger to a first rotation rotates the clamp plate; a firing plate coupled to the push plate, wherein actuation of the closure trigger between the first rotation and a second rotation rotates the firing plate; a cam shaft plate coupled to the closure trigger, wherein the cam shaft plate defines a cam path having a detent; a plunger configured to interface with the cam path, wherein proximal actuation of the closure trigger rotates the cam shaft plate into contact with the plunger, wherein the plunger follows the cam path to interface with the detent, and wherein the detent maintains the cam shaft plate and the closure trigger at a first rotation; a shaft assembly comprising a proximal end and a distal end, wherein the shaft assembly is coupled to the handle assembly at the proximal end; and an end effector coupled to the distal end of the shaft assembly, the end effector comprising: a jaw assembly having a proximal end and a distal end, the jaw assembly comprising: a first jaw member; and a second jaw member, wherein the first and second jaw members define a longitudinal slot, wherein rotation of the clamp plate pivotally moves the first jaw member from an open position to a closed position relative to the second jaw member; and a cutting member deployable within the longitudinal slot, wherein rotation of the firing plate advances the cutting member distally within the longitudinal slot.

What is claimed is:

1. A surgical instrument comprising:
a handle assembly comprising:
a closure trigger;
a push plate coupled to the closure trigger, wherein actuation of the closure trigger rotates the push plate;
a clamp plate coupled to the push plate, wherein actuation of the closure trigger to a first rotation rotates the clamp plate; and
a firing plate coupled to the push plate, wherein actuation of the closure trigger between the first rotation and a second rotation rotates the firing plate;
a shaft assembly comprising a proximal end and a distal end, wherein the shaft assembly is coupled to the handle assembly at the proximal end; and
an end effector coupled to the distal end of the shaft assembly, the end effector comprising:
a jaw assembly having a proximal end and a distal end, the jaw assembly comprising:
a first jaw member; and
a second jaw member, wherein the first and second jaw members define a longitudinal slot, wherein rotation of the clamp plate pivotally moves the first jaw member from an open position to a closed position relative to the second jaw member; and
a cutting member deployable within the longitudinal slot, wherein rotation of the firing plate advances the cutting member distally within the longitudinal slot.

2. The surgical instrument of claim 1, comprising a floating pin coupled to the clamp plate, wherein the floating pin is configured to move within a floating pin path comprising a bypass section, wherein rotation of the closure trigger causes the push plate to move the floating pin within the floating pin path, and wherein the floating pin enters the bypass section and disengages from the push plate to disconnect the push plate from the clamp plate.

3. The surgical instrument of claim 2, wherein the push plate is configured to maintain the floating pin in the bypass section during rotation of the closure trigger from the first rotation to the second rotation.

4. The surgical instrument of claim 3, wherein the clamp plate comprises a toggle clamp.

5. The surgical instrument of claim 4, comprising:
a yoke coupled to the toggle clamp, wherein the toggle clamp is configured to drive to the yoke plate proximally, wherein proximal movement of the yoke transitions the jaws from an open position to a closed position; and
a closure spring, wherein the closure spring is compressed by proximal movement of the yoke.

6. The surgical instrument of claim 1, comprising a trigger pin coupled to the push plate, wherein the trigger pin is configured to move within a trigger pin path defined by the firing plate, wherein the trigger pin path comprises a first section and a second section, wherein rotation of the closure trigger slideably moves the trigger pin within the trigger pin path, wherein the trigger pin moves freely within the first section of the trigger pin path and wherein the trigger pin rotates the firing plate within the second section.

7. The surgical instrument of claim 1, wherein at least one the push plate, the clamp plate, or the firing plate comprises a low-friction material.

8. The surgical instrument of claim 7, comprising:
a rack and pinion coupled to the firing plate, wherein the rack and pinion is configured to pivotally move the first jaw member from an open position to a closed position relative to the second jaw.

9. The surgical instrument of claim 6, wherein a mechanical advantage of the closure trigger changes as the trigger pin traverses the trigger pin path.

10. The surgical instrument of claim 1, comprising:
a cam shaft plate coupled to the closure trigger, wherein the cam shaft plate defines a cam path having a detent; and
a plunger comprising a plunger pin configured to interface with the cam path, wherein proximal actuation of the closure trigger rotates the cam shaft plate into contact with the plunger, wherein the plunger pin follows the cam path to interface with the detent, and wherein the detent maintains the cam shaft plate and the closure trigger at a first rotation corresponding to the first and second jaw members in a closed position.

11. The surgical instrument of claim 10, wherein the detent comprises a first arm and a second arm, and wherein the first arm defines a force required to continue rotation of the closure trigger to fire the cutting member, and wherein the second arm defines a force required to rotate the closure trigger distally to open the jaws.

12. The surgical instrument of claim 10, comprising a toggle switch coupled to the plunger, wherein the toggle switch comprises a first position and a second position, wherein when the toggle pin is in the first position the plunger pin is configured to interface with the cam path, and wherein when the toggle pin is in the second position the plunger pin is configured to bypass the cam path.

13. A surgical instrument comprising:
a handle assembly comprising:
a closure trigger;
a cam shaft plate coupled to the closure trigger, wherein the cam shaft plate defines a cam path having a detent; and
a plunger comprising a plunger pin configured to interface with the cam path, wherein proximal actuation of the closure trigger rotates the cam shaft plate into contact with the plunger pin, wherein the plunger pin follows the cam path to interface with the detent, and wherein the detent maintains the cam shaft plate and the closure trigger at a first rotation;
a shaft assembly comprising a proximal end and a distal end, wherein the shaft assembly is coupled to the handle assembly at the proximal end; and
an end effector coupled to the distal end of the shaft assembly, the end effector comprising:
a jaw assembly having a proximal end and a distal end, the jaw assembly comprising:
a first jaw member; and
a second jaw member, wherein the first and second jaw members define a longitudinal slot, wherein rotation of the clamp plate pivotally moves the first jaw member from an open position to a closed position relative to the second jaw member; and
a cutting member deployable within the longitudinal slot, wherein rotation of the firing plate advances the cutting member distally within the longitudinal slot.

14. The surgical instrument of claim 13, wherein the detent comprises a first arm and a second arm, and wherein the first arm sets a force required to continue rotation of the closure trigger to fire the cutting member, and wherein the second arm sets a force required to rotate the closure trigger distally to open the jaws.

15. The surgical instrument of claim 13, comprising a toggle switch coupled to the plunger, wherein the toggle switch comprises a first position and a second position, wherein when the toggle pin is in the first position the plunger is configured to interface with the cam path, and wherein when the toggle pin is in the second position the plunger is configured to bypass the cam path.

16. The surgical instrument of claim 13, comprising:
a push plate coupled to the closure trigger, wherein actuation of the closure trigger rotates the push plate;
a clamp plate coupled to the push plate, wherein actuation of the closure trigger to a first rotation rotates the clamp plate; and
a firing plate coupled to the push plate, wherein actuation of the closure trigger between the first rotation and a second rotation rotates the firing plate.

17. The surgical instrument of claim 16, comprising a floating pin coupled to the clamp plate, wherein the floating pin is configured to move within a floating pin path comprising a bypass section, wherein rotation of the closure trigger causes the push plate to move the floating pin within the floating pin path, and wherein the floating pin enters the bypass section and disengages from the push plate to disconnect the push plate from the clamp plate.

18. The surgical instrument of claim 17, wherein the push plate is configured to maintain the floating pin in the bypass section during rotation of the closure trigger from the first rotation to the second rotation.

19. The surgical instrument of claim 17, wherein at least one of the plunger pin or the floating pin comprises a low-friction material.

20. A surgical instrument comprising:
a handle assembly comprising:
a closure trigger;
a push plate coupled to the closure trigger, wherein actuation of the closure trigger rotates the push plate;
a clamp plate coupled to the push plate, wherein actuation of the closure trigger to a first rotation rotates the clamp plate;
a firing plate coupled to the push plate, wherein actuation of the closure trigger between the first rotation and a second rotation rotates the firing plate;
a cam shaft plate coupled to the closure trigger, wherein the cam shaft plate defines a cam path having a detent; and
a plunger configured to interface with the cam path, wherein proximal actuation of the closure trigger rotates the cam shaft plate into contact with the plunger, wherein the plunger follows the cam path to interface with the detent, and wherein the detent maintains the cam shaft plate and the closure trigger at a first rotation;
a shaft assembly comprising a proximal end and a distal end, wherein the shaft assembly is coupled to the handle assembly at the proximal end; and
an end effector coupled to the distal end of the shaft assembly, the end effector comprising:
a jaw assembly having a proximal end and a distal end, the jaw assembly comprising:
a first jaw member; and
a second jaw member, wherein the first and second jaw members define a longitudinal slot, wherein rotation of the clamp plate pivotally moves the first jaw member from an open position to a closed position relative to the second jaw member; and
a cutting member deployable within the longitudinal slot, wherein rotation of the firing plate advances the cutting member distally within the longitudinal slot.
